(19) United States
(12) Patent Application Publication
(10) Pub. No.: US 2010/0313339 A1
(43) Pub. Date: Dec. 16, 2010

(54) SWIMMING OR DIVING GOGGLES

(76) Inventor: Terry Chou, Tainan City (TW)

Correspondence Address: Dr. BANGER SHIA;Patent Office of Bang Shia, 102 Lindencrest Ct, Sugar Land, TX, 77479-5201, US

(21) Appl. No.: 12/482,407

(22) Filed: Jun. 10, 2009

(51) Int. Cl. A61F 9/02

(52) U.S. Cl. 2/426

(57) ABSTRACT

Swimming or diving goggles includes a lens unit and a frame mechanism coupled to the lens unit. The lens unit includes two viewing sections each having a front face that is a portion of a peripheral face extending between an apex and a bottom face of a cone. The apex is below the bottom face of the cone. A vertical axis passes through a center of the bottom face of the cone. A spacing from a top edge of each viewing section to the vertical axis in a thickness direction is smaller than a spacing from a bottom edge of the viewing section to the vertical axis in the thickness direction A normal plane perpendicular to each viewing section is at an acute angle with the vertical axis. Each viewing section is a curved face in a circumferential direction about the vertical axis. The swimming or diving goggles is ergonomic and more water-tight.

### BACKGROUND OF THE INVENTION

The present invention relates to swimming or diving goggles and, more particularly, to ergonomic swimming or diving goggles.

Swimming or diving goggles generally include two lenses and a sealing frame. The lenses are rigid and can be separate or integrally formed with each other as a single monolithic piece. The sealing frame is worn on a face of a wearer and in intimate contact with eye sockets of the wearer. The lenses are planar, spherical, or columnar. Planar lenses can not bend to match with the curvature of the face of the wearer and have larger resistance during swimming.

FIGS. 15 and 16 show a conventional spherical lens 1′ that is arcuate in cross section to reduce resistance. However, the curvature of each of front and rear surfaces of the lens 1′ is a portion of a circle, which is not suitable to average users. Furthermore, when worn on a face of a wear having a face contour flatter than the average users, the spacing 1a′ between a center of the face is relatively large such that a center 11′ of the lens 1′ is far away from the face of the wearer.

FIGS. 17 and 18 show a conventional columnar lens 2′. The curvature of the lens 2′ extends toward two sides of the face of the wearer to reduce resistance. A cross section of the lens 2′ taken along a vertical plane includes planar front and rear surfaces such that a center 20′ of the lens 2′ does not protrude like a sphere. However, the lens 2′ is not ergonomic, for the distance 2a′ between the lens 2′ and the forehead of the wearer is smaller than the distance 2b′ between the lens 2′ and the eye socket of the wearer. When pulling a head strap to make the padding members 3′ be in contact with the face of the wearer, the contacting force at the upper portion 3a′ of the face is not equal to that at the lower portion 3b′ of the face, leading to leakage of water at the lower portion 3b′ during swimming or diving.

U.S. Patent Application No. 2004/0064875 discloses swimming or diving goggles including a pair of lenses that are separate or carried out as one piece, and a sealing frame surrounding the lenses. Each lens is provided with a temporal portion and a nasal portion. The nasal portion is bent in a substantially horizontal plane and follows a first theoretical cylindrical surface with substantially vertical generatrixes. The temporal portion is bent in a second theoretical cylindrical surface having a combined bend both on a horizontal plane and on a vertical plane. However, such swimming or diving goggles still have the disadvantages of uneven contacting forces at upper and lower portions of the face of the wearer.

### BRIEF SUMMARY OF THE INVENTION

An objective of the present invention is to provide ergonomic swimming or diving goggles.

Swimming or diving goggles according to the preferred teachings of the present invention includes a lens unit made of rigid material and having two separate lenses or a single monolithic lens. The lens unit includes two viewing sections each having front and rear faces spaced from in a thickness direction. The front face is a portion of a peripheral face of a cone. The cone has an apex and a bottom face. The peripheral face extends between the apex and the bottom face. Each viewing section has top and bottom edges spaced in a vertical direction perpendicular to the thickness direction. The top edge of each viewing section is intermediate the bottom edge of the viewing section and the bottom face of the cone in the vertical direction. The bottom edge of each viewing section is intermediate the top edge of the viewing section and the apex of the cone in the vertical direction. A vertical axis passes through a center of the bottom face of the cone. A spacing from the top edge of each viewing section to the vertical axis in the thickness direction is smaller than a spacing from the bottom edge of the viewing section to the vertical axis in the thickness direction A normal plane perpendicular to each viewing section is at an acute angle with the vertical axis. Each viewing section is a curved face in a circumferential direction about the vertical axis. A frame mechanism is coupled to the lens unit. The frame mechanism is adapted to be worn on a wearer wearing the swimming or diving goggles and in intimate contact with eye sockets of the wearer with a spacing between the top edge of each viewing section and a face of the wearer in the thickness direction is smaller than that between the bottom edge of each viewing section and the face of the wearer in the thickness direction. The swimming or diving goggles is ergonomic and more water-tight.

The present invention will become clearer in light of the following detailed description of illustrative embodiments of this invention described in connection with the drawings.

### DESCRIPTION OF THE DRAWINGS

The illustrative embodiments may best be described by reference to the accompanying drawings where:

FIG. 1 shows a perspective view of a lens unit of swimming or diving goggles of a first embodiment according to the preferred teachings of the present invention.

FIG. 2 shows a cross sectional view of the lens unit of FIG. 1 according to section line A-A in FIG. 1.

FIG. 3 shows a cross sectional view of the lens unit of FIG. 1 according to section line B-B in FIG. 1.

FIG. 4 shows a perspective view of the swimming or diving goggles including the lens unit of FIG. 1.

FIG. 5 shows a cross sectional view of the swimming or diving goggles of FIG. 4 worn on a wearer.

FIG. 6 shows a perspective view of a lens unit of swimming or diving goggles of a second embodiment according to the preferred teachings of the present invention.

FIG. 7 shows a perspective view of a lens unit of swimming or diving goggles of a third embodiment according to the preferred teachings of the present invention.

FIG. 8 shows a front view of the swimming or diving goggles of FIG. 7 worn on a face of a wearer.

FIG. 9 shows a top view of the swimming or diving goggles of FIG. 7 worn on the wearer.

FIG. 10 shows a perspective view of a lens unit of swimming or diving goggles of a fourth embodiment according to the preferred teachings of the present invention.

FIG. 11 shows a perspective view of a lens unit of swimming or diving goggles of a fifth embodiment according to the preferred teachings of the present invention.

FIG. 12 shows a perspective view of a lens unit of swimming or diving goggles of a sixth embodiment according to the preferred teachings of the present invention.

FIG. 13 shows a perspective view of a lens unit of swimming or diving goggles of a seventh embodiment according to the preferred teachings of the present invention.

FIG. 14 shows a partial, perspective view of a lens unit of swimming or diving goggles of an eighth embodiment according to the preferred teachings of the present invention.

FIG. 15 shows a cross sectional view of a conventional lens.

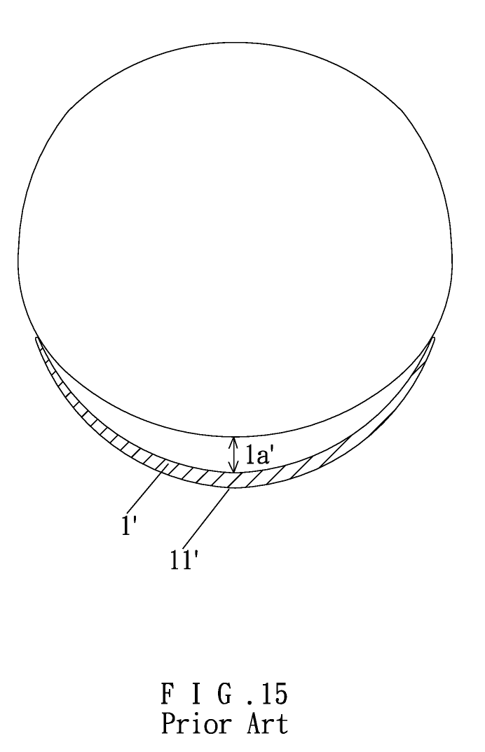

FIG. 16 shows another cross sectional view of the conventional lens of FIG. 15 worn on a face of a wearer.

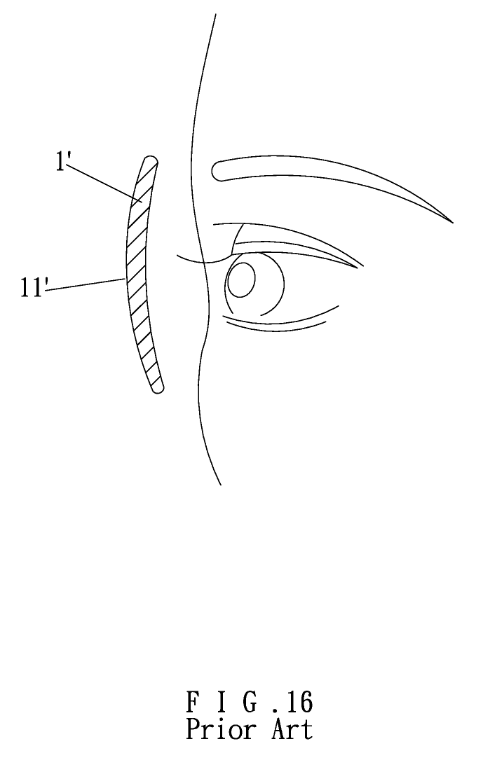

FIG. 17 shows a cross sectional view of another conventional lens.

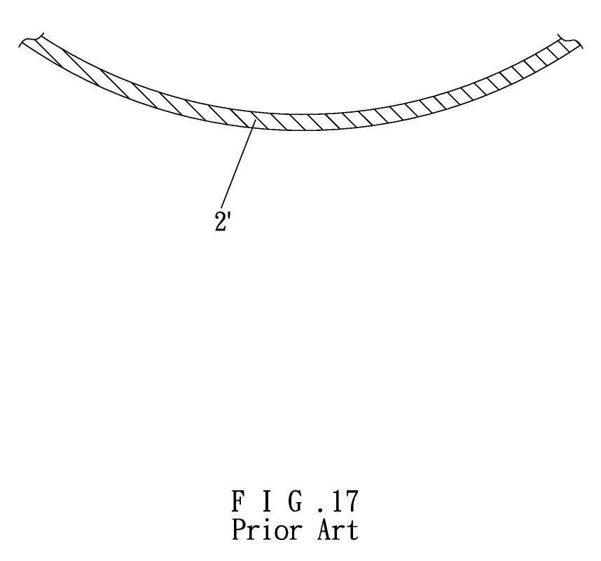

FIG. 18 shows another cross sectional view of the conventional lens of FIG. 17 worn on a face of a wearer.

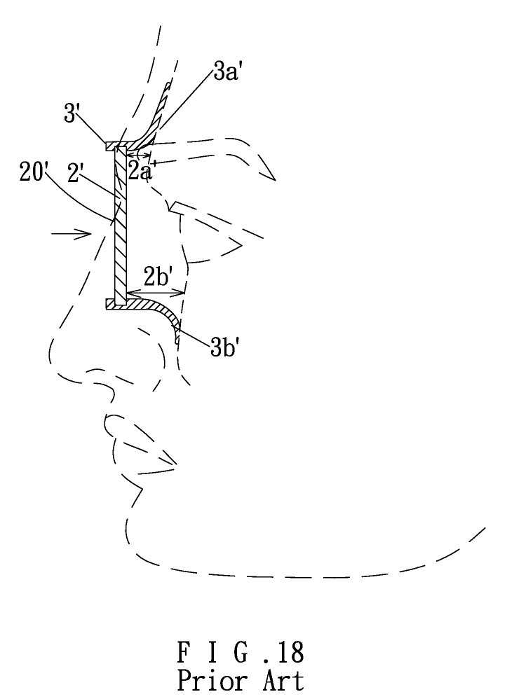

### DETAILED DESCRIPTION OF THE INVENTION

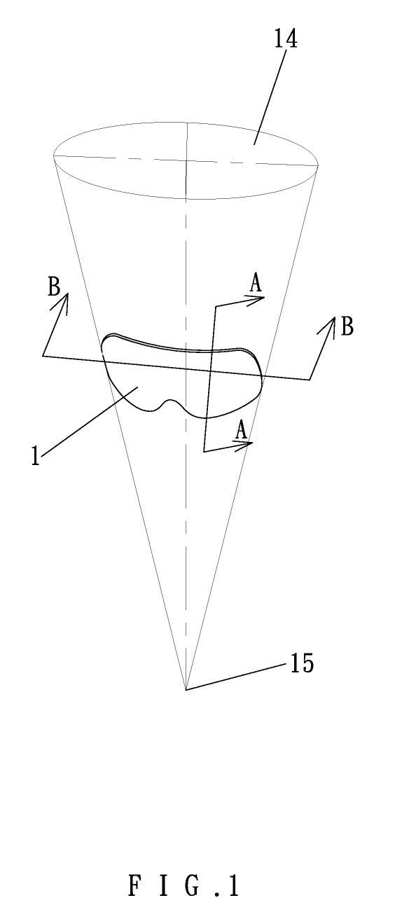

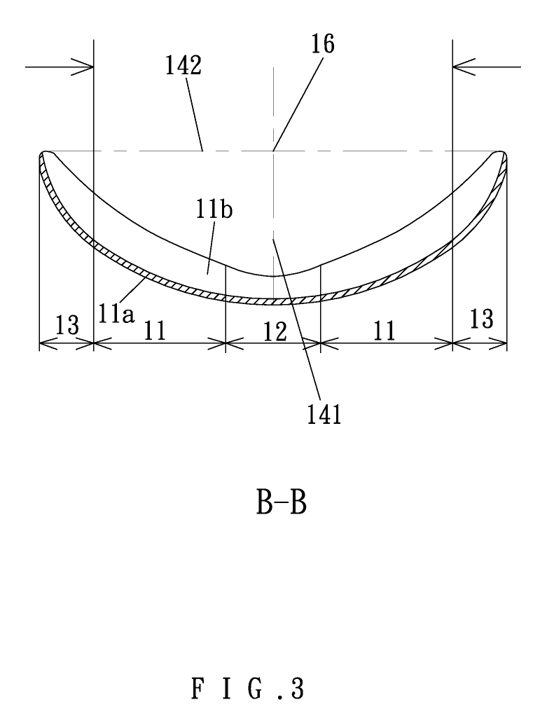

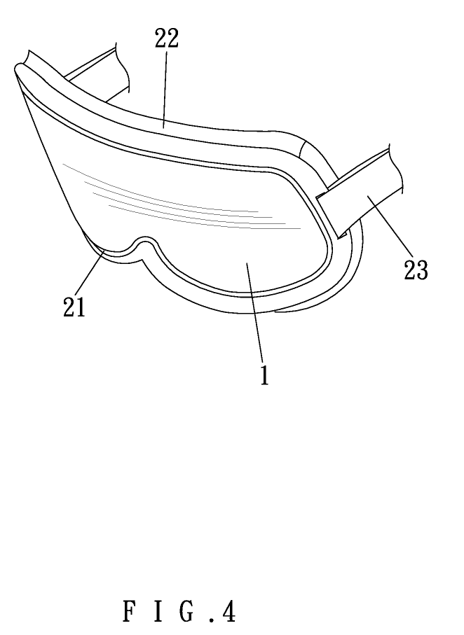

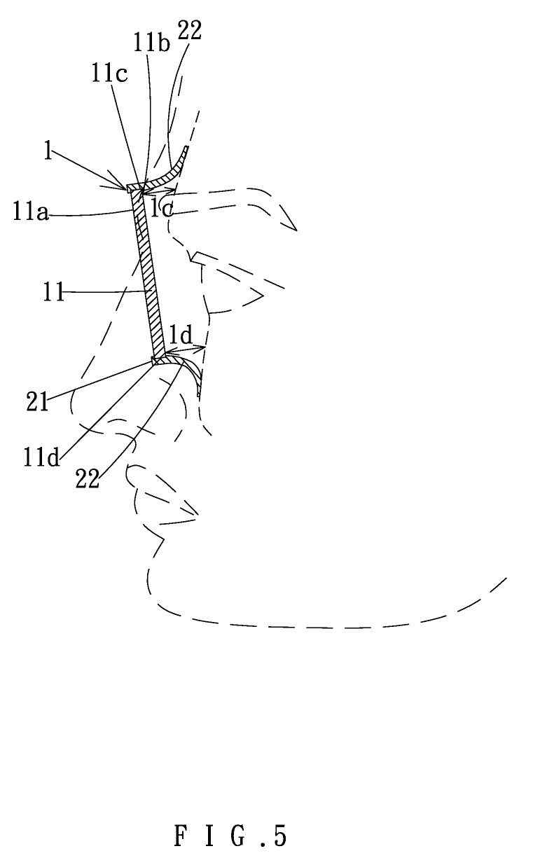

FIGS. 1-5 show swimming or diving goggles of a first embodiment according to the preferred teachings of the present invention. In this embodiment, swimming or diving goggles includes a lens unit 1 in the form of a single monolithic lens and a frame mechanism 2. The lens unit 1 is made of rigid material and includes a nasal section 12, two viewing sections 11 on opposite sides of the nasal section 12, and two side sections 13 on opposite sides of the viewing sections 11. Each viewing section 11 is intermediate the nasal section 12 and one of the side sections 13 in a width direction. Each viewing section 11 corresponds to an area between a nose and one of two temples of a wearer. The nasal section 12 corresponds to a location of the nose of the wearer. Each side section 13 corresponds to a location of one of the temples of the wearer. Each viewing section 11 includes front and rear faces 11a and 11b spaced in a thickness direction perpendicular to the width direction. Each viewing section 11 further includes top and bottom edges 11c and 11d spaced in a vertical direction perpendicular to the width and thickness directions. The front face 11a is a portion of a three-dimensional shape of a cone having a bottom face 14, an apex 15 below the bottom face 14, and a peripheral face extending between the apex 15 and the bottom face 14. The top edge 11c is intermediate the bottom edge lid and the bottom face 14 of the cone in the vertical direction. The bottom edge lid is intermediate the top edge 11c and the apex 15 in the vertical direction. The cone includes elliptical cross sections with a vertical axis 16 passing through the apex 15 and a center of the bottom face 14 being perpendicular to the bottom face 14 that is an ellipse. The front face 11a is a portion of the peripheral face of the cone. Furthermore, the front face each of the nasal section 12 and the side sections 13 is also a portion of the peripheral face.

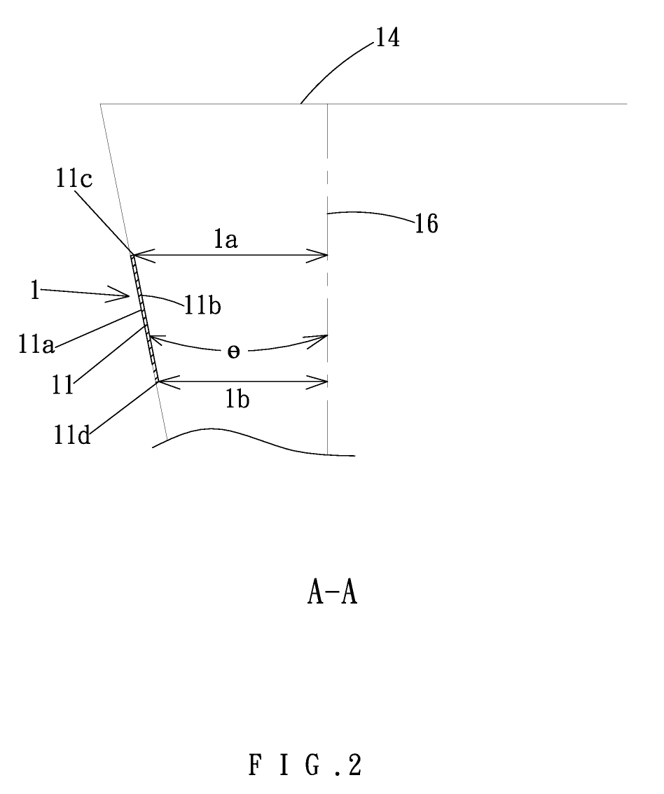

The top edge 11c of each viewing section 11 has a spacing la to the vertical axis 16 in the thickness direction smaller than a spacing 11b between the bottom edge 11d of the viewing section 11 and the vertical axis 16. Furthermore, a normal plane perpendicular to each viewing section 11 is at an acute angle θ with the vertical axis 16 (FIG. 2). The viewing sections 11, the nasal section 12, and the side sections 13 together form a curved face in a circumferential direction about the vertical axis 16. The nasal section 12 and the viewing sections 11 are at an end portion of a shorter axis 141 of an ellipse. The side sections 13 are at two ends of a longer axis 142 of the ellipse. Thus, each side section 13 has a curvature larger than those of the viewing sections 11 and the nasal section 12. The side sections 13 bend toward two sides of a head of the wearer, providing an ergonomic, water-tight design.

The frame mechanism 2 includes an annular frame 21 mounted around and receiving the lens unit 1. The frame mechanism 2 further includes a padding member 22 extending rearward from an inner face of the annular frame 21 for intimate contact with eye sockets of a wearer. The frame mechanism 2 further includes a head strap 23 coupled to two sides of the annular frame 21. When worn on the head of the wearer, the spacing between the top edge 11c of each viewing section 11 and the face of the wearer in the thickness direction is smaller than that between the bottom edge 11d of each viewing section 11 and the face of the wearer in the thickness direction. Furthermore, the vertical cross section of the lens unit 1 is planar such that a center of the lens unit 1 does not protrude far away from the face of the wearer. Furthermore, the difference between a spacing 1c between the forehead of the wearer and the rear face 11b of each viewing section 11 in the thickness direction and a spacing 1d between each eye socket of the wearer and the rear face 11b of one of the viewing sections 11 is relatively small and, thus, ergonomic. Since the horizontal cross section of the lens unit 1 is curved, resistance to the lens unit 1 is small when the wearer moves.

The smaller the distance between the apex 15 and the bottom face 14 of the cone is, the larger the acute angle θ is. This design allows easy mass production. Since the curved face of the lens unit 1 is a portion of a peripheral face of a cone, an ergonomic design suitable for differing wearers can be easily obtained. Note that the lens unit 1 is suitable for Asians.

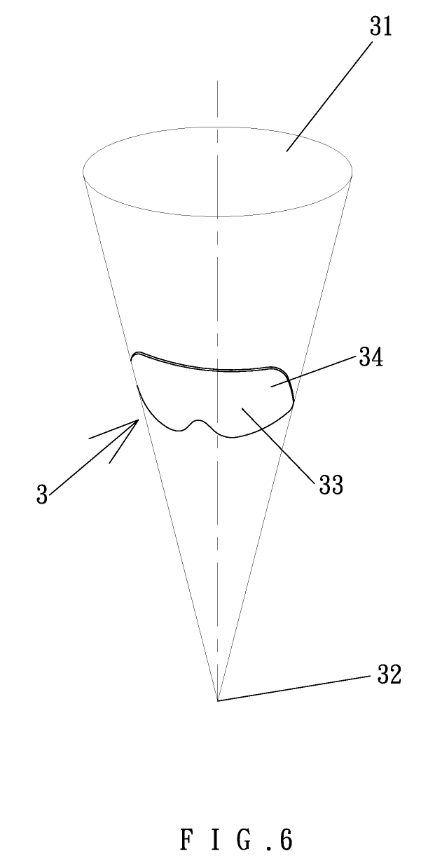

FIG. 6 shows a second embodiment of the present invention, wherein the cone includes an apex 32 and a bottom face 31 and includes circular cross sections such that the side sections 34 and the viewing sections 33 have the same curvature. Note that the lens unit 1 of this embodiment is suitable for westerns.

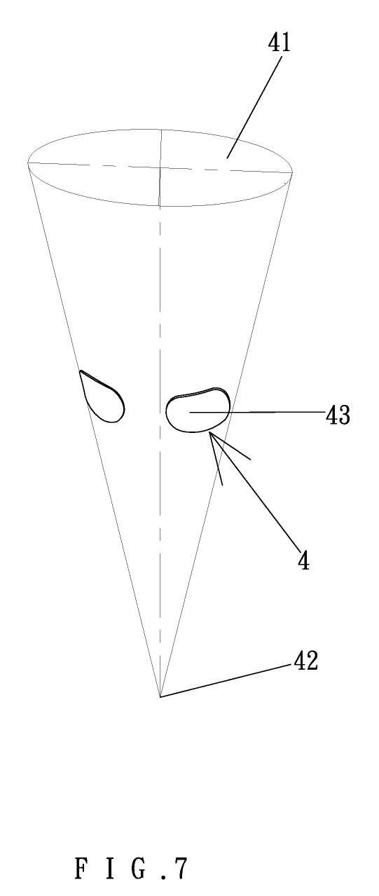

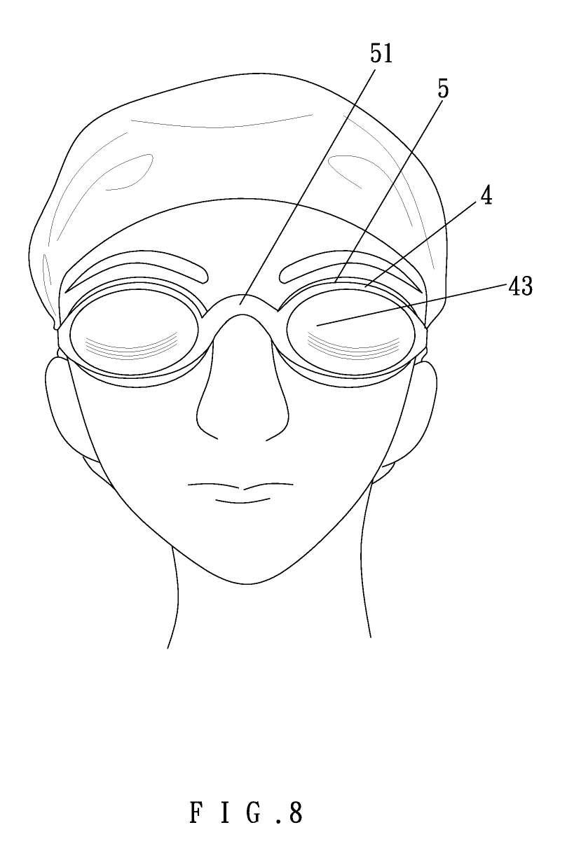

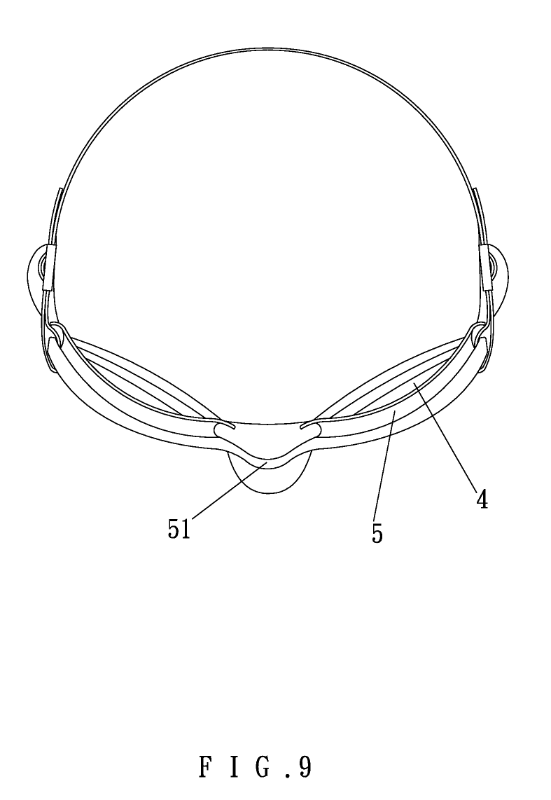

FIGS. 7-9 show a third embodiment of the present invention, wherein the lens unit includes two separate lenses 4. Each lens 4 is a portion of the peripheral face of the cone. The viewing section 43 of each lens 4 corresponds to the area between the nose and one of the temples of the wearer. The lenses 4 are received in a frame mechanism 5 having a bridge 51. The lenses 4 are aligned with the eye sockets of the wearer when the swimming or diving goggles is worn on a face of the wearer, providing a visual angle the same as that in the first embodiment.

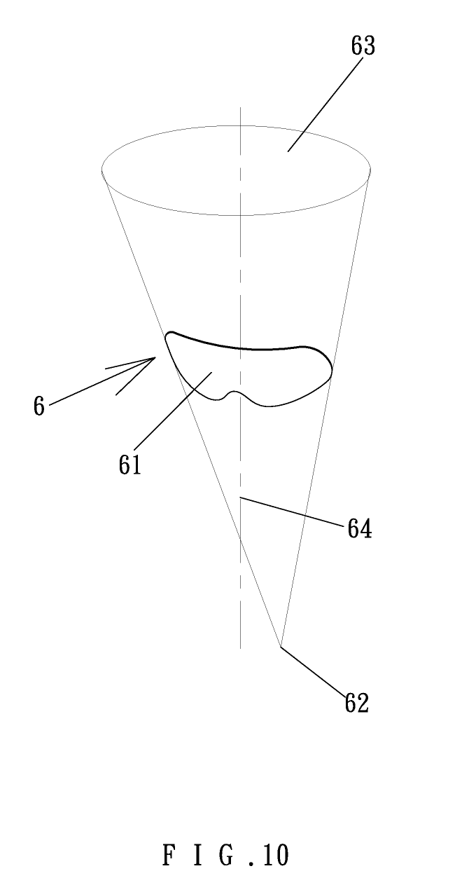

The cone can be varied to include differing curvatures and differing slopes according to design needs. FIG. 10 shows a fourth embodiment of the present invention. In this embodiment, each viewing section 61 of the lens unit 6 is a portion of a peripheral face of a cone having an apex 62 and a bottom face 63. However, the apex 62 is not located in the vertical axis 64 passing through the center of the bottom face 63.

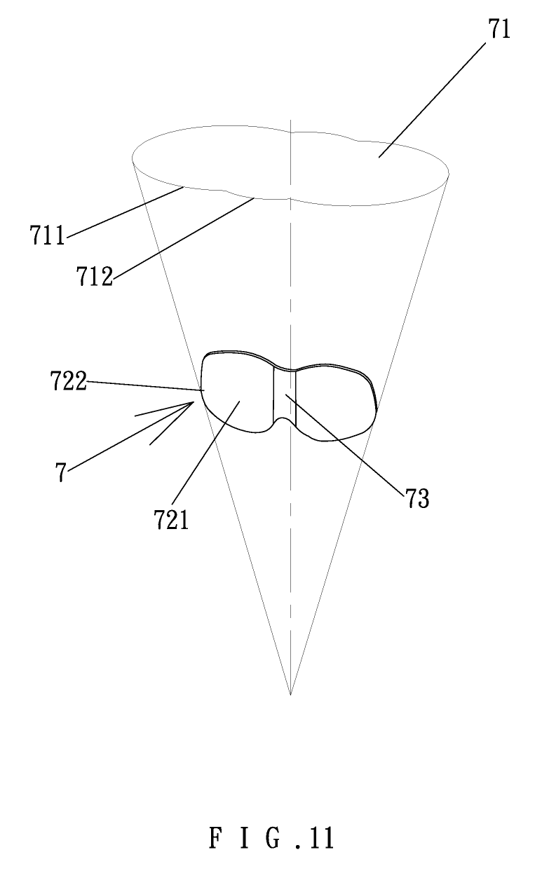

FIG. 11 shows a fifth embodiment of the present invention. In this embodiment, the cone includes a bottom face 71 having two arcuate portions 711 and two bulge portions 712. The lens 7 includes a nasal section 73, two viewing sections 721 and two side sections 722. The arcuate portions 711 correspond to the viewing sections 721 and the side sections 722. One of the bulge portions 712 corresponds to the nasal section 73 to reduce pressing force imparted to the nose of the wearer.

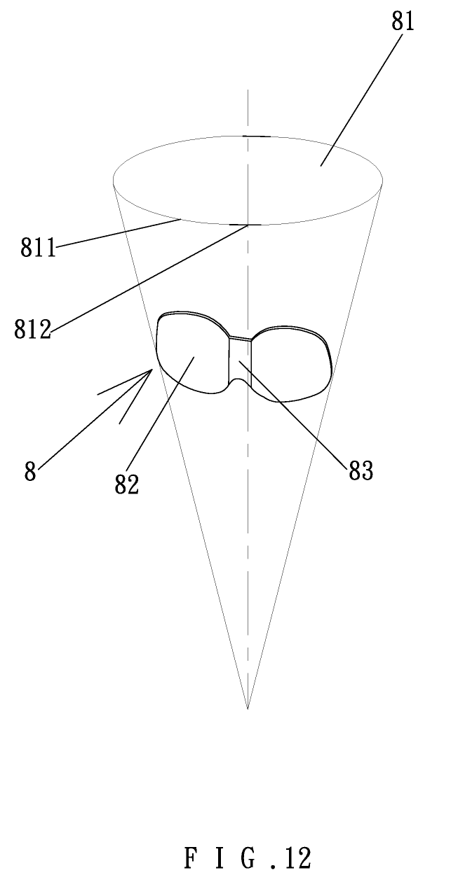

FIG. 12 shows a sixth embodiment of the present invention, wherein the cone includes a bottom face 81 having two arcuate portions 811 and two rectilinear portions 812. The arcuate portions 811 correspond to the viewing sections 82 of the lens 8. One of the rectilinear portions 812 corresponds to the nasal section 83 of the lens 8. The nasal section 83 will not interfere with the visual field during use.

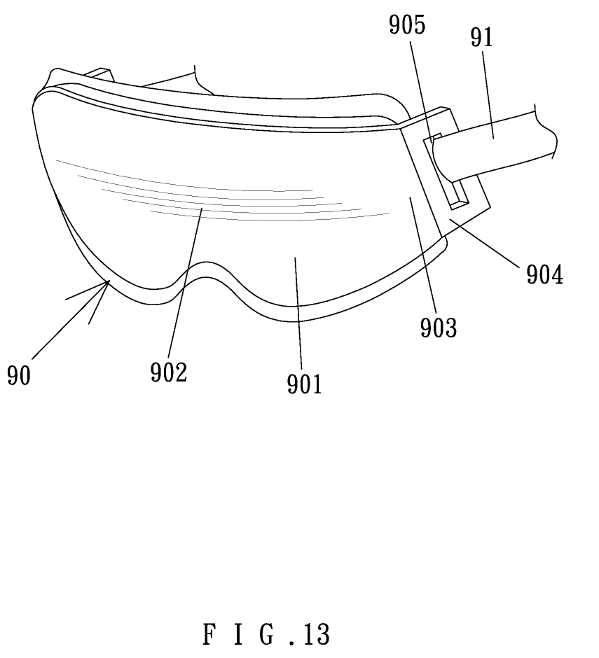

The lens unit according to the teachings of the present invention can include an extension extending from the viewing sections, the nasal section, or the side sections without adversely affecting the visual field. Such an extension does not have to be a part of the peripheral face of the cone. In an example shown in FIG. 13 showing a seventh embodiment of the present invention, the lens unit 90 includes two viewing sections 901, a nasal section 902, and two side sections 903. Each viewing section 901 is a portion of a peripheral face of a cone. Extensions 904 extend from the side sections 903 and each include an opening 905 for coupling with a frame mechanism 91.

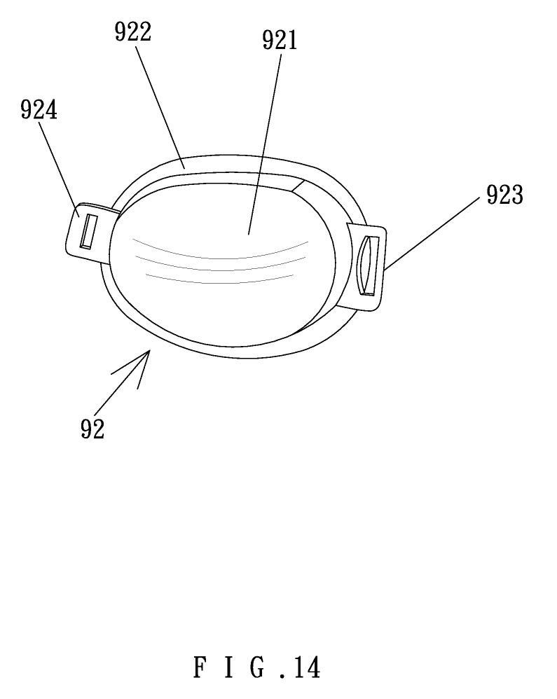

FIG. 14 shows an eighth embodiment of the present invention, wherein the lens unit includes two separate lenses 92 (only one is shown) each having an extension 922 for coupling with a padding member, a head coupling section 923 for coupling with an end of a head strap, and a bridge coupling section 924 for coupling with a bridge.

Thus since the invention disclosed herein may be embodied in other specific forms without departing from the spirit or general characteristics thereof, some of which forms have been indicated, the embodiments described herein are to be considered in all respects illustrative and not restrictive. The scope of the invention is to be indicated by the appended claims, rather than by the foregoing description, and all changes which come within the meaning and range of equivalency of the claims are intended to be embraced therein.

1. Swimming or diving goggles comprising:
a lens unit made of rigid material and including two separate lenses or a single monolithic lens, with the lens unit including two viewing sections each having front and rear faces spaced from in a thickness direction, with the front face being a portion of a peripheral face of a cone, with the cone having an apex and a bottom face, with the peripheral face extending between the apex and the bottom face, with each of the two viewing sections having top and bottom edges spaced in a vertical direction perpendicular to the thickness direction, with the top edge of each of the two viewing sections intermediate the edge face of the viewing section and the bottom edge of the cone in the vertical direction, with the bottom edge of each of the two viewing sections intermediate the top edge of the viewing section and the apex of the cone in the vertical direction, with a vertical axis passing through a center of the bottom face of the cone, with a spacing from the top edge of each of the two viewing sections to the vertical axis in the thickness direction being smaller than a spacing from the bottom edge of the viewing section to the vertical axis in the thickness direction, with a normal plane perpendicular to each of the two viewing sections being at an acute angle with the vertical axis, with each of the two viewing sections being a curved face in a circumferential direction about the vertical axis; and
a frame mechanism coupled to the lens unit, with the frame mechanism adapted to be worn on a wearer wearing the swimming or diving goggles and in intimate contact with eye sockets of the wearer with a spacing between the top edge of each of the two viewing sections and a face of the wearer in the thickness direction is smaller than that between the bottom edge of each of the two viewing sections and the face of the wearer in the thickness direction.

2. The swimming or diving goggles as claimed in claim 1, with each of the two viewing sections corresponding to an area between a nose and one of two temples of the wearer.

3. The swimming or diving goggles as claimed in claim 2, with the lens unit further including two side sections on opposite sides of the two viewing sections, with each of the side sections corresponding to a location of one of the temples of the wearer.

4. The swimming or diving goggles as claimed in claim 2, with the lens unit being a single monolithic lens including the two viewing sections, with the single monolithic lens further including a nasal section intermediate the two receiving sections, and with the nasal section corresponding to a location of the nose of the wearer.

5. The swimming or diving goggles as claimed in claim 3, with the bottom face being an ellipse, with the cone including elliptic cross sections each having a shorter axis and a longer axis, with the two viewing sections being on an end portion of the shorter axis, with the side sections being on two ends of the longer axis, and with each of the two side sections having a curvature larger than that of the two viewing sections.

6. The swimming or diving goggles as claimed in claim 4, with the bottom face including two arcuate portions and two bulge portions, and with the two arcuate portions corresponding to the two viewing sections, with one of the two bulge portions corresponding to the nasal section.

7. The swimming or diving goggles as claimed in claim 1, with the lens unit further including an extension, with the extension being not a portion of the peripheral face of the cone.

8. The as claimed in claim 8, with the extension coupled to the frame mechanism.

9. The as claimed in claim 1, with the lens unit further including an extension, with the extension being a portion of the peripheral face of the cone.

10. The as claimed in claim 9, with the extension coupled to the frame mechanism.
